SEQUENCE LISTING (31 sequences)

SEQ ID NO: 1 - protein, 363 aa, Chlorella virus NC64A
MRTKYRIFIVCMLMSMKALELFAGIGGITHGLRGYVEPIAFCEYEKDASSFLSQRGLPVHGDITKFDASVYKNKIDIVTAGWPCTGFSTAGKGTGFEHEASGLWTEVVRVVKESEPKYVFLENSHVLAQTKNLKVIIHDLDILGYDTRWWTCRSNDVNVGAHHNRYRWFMLAEKKGSVTKFVKIQVKKFNWSGDFKEKQISENSHENKQLIKFMGNSVVPDQVRYAFESMSDMILEGSLVNDKDEIVKVGYSKDGIMYKIPIEHKIIPKLNIVLTPRDPPEGHKAREEAIIKSPILMTYWNTPAFCYHKSARGAKILTKRQKNNLHTQIKFCPGGSDDGYLSGRFCAWLMGYDDEYLGDLMHY

SEQ ID NO: 2 - protein, 362 aa, Chlorella virus NC64A
MTLKALELFAGIAGITHGLRGFVEPVAFVEINKDAQEFLSTKFPDKPVFDDVTKFSKRDFDEPIDMITGGFPCTGFSIAGKRNGFEHAESGLFGEVVRITKEYMPKMVFLENSGMLSHKYNLDIVIRSMDSLGYDCRWVTLRATVVGALHTRHRWFCLCTRKDHIRETLICDREVTKFDWENDRPPIQVDSRSYENSRLVRFAGYSVVPDQIRYAFTGLYTGNFSPSFSKTLVPGSLEGSICFNEDKITNGYYKDGVYYEFVRTETHREPVNILLTPREIPNKHNGKKLLTLPVTKRYWCTPCASYGKGTAGGRVLTDRSSHSLPTQVKFSPEGEDGKHLSGKFCAWLMGYDKEYLGNLLEY

SEQ ID NO: 3 - protein, 327 aa, Haemophilus haemolyticus
MIEIKDKQLTGLRFIDLFAGLGGFRLALESCGAECVYSNEWDKYAQEVYEMNFGEKPEGDITQVNEKTIPDHDILCAGFPCQAFSISGKQKGFEDSRGTLFFDIARIVREKKPKVVFMENVKNFASHDNGNTLEVVKNTMNELDYSFHAKVLNALDYGIPQKRERIYMICFRNDLNIQNFQFPKPFELNTFVKDLLLPDSEVEHLVIDRKDLVMTNQEIEQTTPKTVRLGIVGKGGQGERIYSTRGIAITLSAYGGGIFAKTGGYLVNGKTRKLHPRECARVMGYPDSYKVHPSTSQAYKQFGNSVVINVLQYIAYNIGSSLNFKPY

SEQ ID NO: 4 - protein, 356 aa, Chlorella virus NC64A
MYIYMSTPQAKTKYYEQRFVNDFYKELERNKVSLPVTIVLKDNLGIKQVIQNVSGVRVLRDKANAKSPSKIKSEELGRHVTSKADIALFTEEKNGTKVDVAWISHKSNKDIHGKKITHAQYLDASSDVMFKTKIGQTKEIKDFKNKMISLSVPLTATKYCWPKYKSGTSLRIWDDVKSTILMNMAIFGVEFGKAYSRNNANILMGGDPLIEVKDDKTIILTTKENGFSLANGFAEYIPSKDKPIFFTKPTSGKKTVVDGKTIEGVSVWIIYRSYAGSKNRKIDDVLKNKIELISSSCSVKKKDNFVSIMQSKKITSPPKSKKITSPPKSKKITSPSKSKKITNFFMKKLKYSLLSR

SEQ ID NO: 5 - protein, 278 aa, Chlorella virus NC64A
MEEKKRLALIEKQRIAEEKIASGRKIRKRISTNATKHEREFVKVINSMFVGPATFVFVDIKGNKSREIHNVVRFRQLQGSKAKSPTAYVDREYNKPKADIAAVDITGKDVAWISHKASEGYQQYLKISGKNLKFTGKELEEVLSFKRKVVSMAPVSKIWPANKTVWSPIKSNLIKNQAIFGFDYGKKPGRDNVDIIGQGRPIITKRGSILYLTFTGFSALNGHLENFTGKHEPVFYVRTERSSSGRSITTVVNGVTYKNLRFFIHPYNFVSSKTQRIM

SEQ ID NO: 6 - protein, 11 aa, Chlorella virus NC64A
MSTPQAKTKYY

SEQ ID NO: 7 - DNA, 49 bp, unknown
  primer
cggtaagcsatgcatgcgtacaaagtatcgtatttttatcgtatgtatg

SEQ ID NO: 8 - DNA, 10 bp, Chlorella virus NC64A
tacggccggg

SEQ ID NO: 9 - DNA, 10 bp, Chlorella virus NC64A
  misc_feature (7)..(7): n is a, c, g, or t
tatggcnggg

SEQ ID NO: 10 - DNA, 10 bp, Chlorella virus NC64A
atggccatgt

SEQ ID NO: 11 - DNA, 10 bp, Chlorella virus NC64A
atggctatgt

SEQ ID NO: 12 - DNA, 15 bp, Chlorella virus NC64A
cctgatcgggaacta

SEQ ID NO: 13 - DNA, 15 bp, Chlorella virus NC64A
  misc_feature (4)..(4): n is a, c, g, or t
cctnatcnggaacta

SEQ ID NO: 14 - DNA, 11 bp, Chlorella virus NC64A
gaaccaaaata

SEQ ID NO: 15 - DNA, 11 bp, Chlorella virus NC64A
gaaccaaaata

SEQ ID NO: 16 - DNA, 17 bp, Chlorella virus NC64A
gatacttgtggcgaatg

SEQ ID NO: 17 - DNA, 12 bp, unknown
  primer
gatacttgtgga

SEQ ID NO: 18 - DNA, 59 bp, Chlorella virus NC64A
  misc_feature (24)..(24): n is a, c, g, or t
gatacgtcgtaagatacttgtggngaatgcgngcgataagcttggcgtaatcatggtcg

SEQ ID NO: 19 - DNA, 60 bp, Chlorella virus NC64A
cgatacgtcgtaagatacttgcggcgaatgcaagcgataagcttggcgtaatcatggccc

SEQ ID NO: 20 - DNA, 59 bp, Chlorella virus NC64A
  misc_feature (23)..(23): n is a, c, g, or t
cgatacgtcgtaagatacttggngcgaatgcaagcgataagcttgncgtantcatggac

SEQ ID NO: 21 - DNA, 62 bp, Chlorella virus NC64A
gatacgtcgtaagatcttgaggcgaatgagcgataagcttggcgtaatcatggacatagctg

SEQ ID NO: 22 - DNA, 43 bp, unknown
  primer
gaagtcgacttaataatgcataagatcacccaaatattcgtcg

SEQ ID NO: 23 - DNA, 45 bp, unknown
  primer
accgttgagcatatgtatatatatatgtctactccgcaggcaaag

SEQ ID NO: 24 - DNA, 66 bp, unknown
  primer
ggtggttgctcttccgcaacaggaagaagaaattaattctattttatttttcaaaacatcatcgat

SEQ ID NO: 25 - DNA, 54 bp, unknown
  primer
ggtggttgctcttccgcagcattttggtggacttgttattttctttgattttgg

SEQ ID NO: 26 - DNA, 20 bp, unknown
  primer
tgcmtcctgcaccaccaact

SEQ ID NO: 27 - DNA, 19 bp, unknown
  primer
ygcctgcttcaccaccttc

SEQ ID NO: 28 - DNA, 1047 bp, Chlorella virus NC64A
atgtatatatatatgtctactccgcaggcaaagaccaaatattatgaacaaagattcgtaaacgatttctacaaagaactcgaacgtaataaagtttctctgcctgtcacaatagttttgaaggataatctgggaataaaacaagttatacagaatgggagtggtgttagagttttacgtgataaagctaatgcgaaatctccgtctaagataaaaagtgaagaattaggtagacatgtgacatcgaaagcggatatagcattatttaccgaggaaaagaatggaacaaaagttgacgttgcatggatatccccccaatctcataaagattttcttggaaaaaagataactcctgctcaatattttgatgcctcctcagatgttatgtttaaaaccaaaattggccaaccaaaggaaataaaggagcttaagaataagatgatttccttgagtgttcctctcacggcaacgaaatattgctggcccaagtacaaatctggaacatctctcagaatatgggacgatgtacaaagcactatacttatgaacatggcaatatttggtgttgagtttggaaaggcatattgtcgaaataatgcaaatattttgatggttggagatcctcttatagaagtaaaagatgacaaaacgataattcttaccacaaaagaaaatggatttagtctggcaaacggatttgcagaatatataccatcaaaggataaacctatatttttcactaaaccaacatcaggaaaaaaaacagtcgtagatggaaaaacgatcgagggagtgagtgtatggataatttatagaagttatgcaggatcaaagaatagaaaaatcgatgatgttttgaaaaataaaatagaattaatttcttcttcctgttctgttaaaaagaaagataactttgtttctatcatgcaatcaaagaaaataacaagtccaccaaaatcaaagaaaataacaagtccaccaaaatcaaagaaaataacaagtccatcaaaatcaaagaaaataacaaatttttttatgaaaaaataa

SEQ ID NO: 29 - protein, 348 aa, Chlorella virus NC64A
MYIYMSTPQAKTKYYEQRFVNDFYKELERNKVSLPVTIVLKDNLGIKQVIQNGSGVRVLRDKANAKSPSKIKSEELGRHVTSKADIALFTEEKNGTKVDVAWISPQSHKDFLGKKITPAQYFDASSDVMFKTKIGQPKEIKELKNKMISLSVPLTATKYCWPKYKSGTSLRIWDDVQSTILMNMAIFGVEFGKAYCRNNANILMVGDPLIEVKDDKTIILTTKENGFSLANGFAEYIPSKDKPIFFTKPTSGKKTVVDGKTIEGVSVWIIYRSYAGSKNRKIDDVLKNKIELISSSCSVKKKDNFVSIMQSKKITSPPKSKKITSPPKSKKITSPSKSKKITNFFMKK

SEQ ID NO: 30 - DNA, 1092 bp, Chlorella virus NC64A
atgcgtacaaagtatcgtatttttatcgtatgtatgttaatgagtatgaaagcattagaactcttcgcaggtataggcgggatcactcatggtcttcgtggttacgtggaaccaattgctttttgtgaatatgaaaaagacgcgtcatcttttttgagtcaacgtggccttccggttcacggagatatcacgaaattcgatgcttctgtgtacaaaaacaagatcgatatcgttaccgctggatggccatgtaccggtttcagtacggccgggaaaggaactggtttcgaacatgaagcgtccggtttatggacggaagtcgtcagagtggtcaaagaaagcgaaccaaaatatgtttttctcgagaattctcatgtgttagctcagacaaaaaatctcaaagtaatcattcacgatctcgacattttgggatatgatactcgttggtggacgtgtcgttcgaacgatgtcaatgtaggcgcacatcacaacagatatagatggttcatgctcgccgaaaagaaaggttctgtgacaaagttcgttaaaattcaagtgaaaaaatttaattggtctggtgatttcaaagagaaacaaatttcagaaaattctcatgaaaacaaacaactcataaagtttatgggaaatagtgtagttcccgatcaggttcgatacgctttcgaatcgatgagcgatatgattctcgaaggatctctcgtgaacgataaggacgaaatcgtgaaagtaggatattctaaagatggtatcatgtacaagataccaatagaacataagattattccaaaacttaatatcgtattgacgcctcgtgatcctccggaaggtcacaaagctcgagaagaagcaatcatcaaatctcctatattgatgacttactggaatactcctgcattctgttatcacaaatccgcacgaggagcaaaaattctcacaaaacgacagaagaataacttacatacacaaattaaattctgtcctggaggttccgatgatggttacttgtctggaagattttgcgcatggctcatgggatatgacgacgaatatttgggtgatcttatgcattattaa

SEQ ID NO: 31 - protein, 363 aa, Chlorella virus NC64A; identical to SEQ ID NO: 1

(19) United States
(12) Patent Application Publication
(10) Pub. No.: US 2008/0268507 A1
(43) Pub. Date: Oct. 30, 2008

(54) Recombinant Dna Nicking Endonuclease and Uses Thereof

(75) Inventors: Shuang-yong Xu, Lexingston, MA (US); Zhenyu Zhu, Beverly, MA (US); Shi-hong Chan, Ipswich, MA (US); Yan Xu, Hamilton, MA (US)

Correspondence Address: HARRIET M. STRIMPEL, D. Phil., New England Biolabs, Inc., 240 COUNTY ROAD, IPSWICH, MA, 01938-2723, US

(73) Assignee: AIRBUS DEUTSCHLAND GMBH, HAMBURG GERMANY (DE)

(21) Appl. No.: 11/666,148

(22) PCT Filed: Oct. 21, 2005
(86) PCT No.: PCT/US05/37607

§ 371 Date: Jan. 29, 2008

Related U.S. Application Data
(60) Provisional application No. 60/620,939, filed on Oct. 21, 2004.

(30) Foreign Application Priority Data
May 25, 2004 (DE) 102004025377.3

(51) Int. Cl. C12P 19/34; C07H 21/04; C12N 9/16; C12N 9/10; C12N 15/00; C12N 1/21

(52) U.S. Cl. 435/91.2; 536/23.1; 536/23.2; 435/196; 435/193; 435/320.1; 435/252.3

(57) ABSTRACT

Recombinant nicking endonucleases and associated methylases have been obtained and sequenced and their specificity has been defined. A mutant form of the nicking endonuclease has been cloned where the mutation includes deletion of amino acid sequences at the C-terminal end of the protein. The nicking enzymes have been used for a number of purposes including: amplifying DNA from as few cells as can be found in a single bacterial colony in the presence of a strand displacing polymerase; and for removing genomic DNA in a biological preparation where it is deemed to be a contaminant.

### BACKGROUND OF THE INVENTION

DNA nicking endonucleases cleave one strand of DNA in a sequence-specific and strand-specific manner. Although there are over 240 type II restriction endonucleases with unique specificities isolated from bacterial and viral sources, only a few site-specific nicking endonucleases are currently commercially available (Roberts et al. Nucl. Acids Res. 31:418-420 (2003); REBASE). Efforts to develop more nicking endonucleases consist of either genetic engineering from existing restriction endonucleases or screening from bacterial and viral sources. The nicking endonuclease N.BstNBI and N.BstSEI (5′ GAGTCN₄^{A} 3′) were found in strains of Bacillus stearothermophilus (Morgan et al. Biol. Chem. 381: 1123-1125 (2000); Abdurashitov et al. Mol. Biol. (Mosk) 30: 1261-1267 (1996)) and the nicking endonuclease N.BsrDI (5′^{Λ} CATTGC 3′), the large subunit of BsrDI, was found in the strain B. stearothermophilus D70. Two natural nicking endonucleases Nt.CviPII (̂CCD) and Nt.CviQXI (R^{Λ} AG) from chlorella viruses have been described (Xia et al. Nucl. Acids Res. 16:9477-87 (1988); Zhang et al. Virology, 240: 366-75 (1998)).

The low quantities of Nt.CviPII in NYs-I infected lysate limited the potential application of this nicking endonuclease in DNA manipulation. To overcome this limitation, it would be desirable to clone and express this nicking-modification system.

### SUMMARY OF THE INVENTION

In an embodiment of the invention, an isolated DNA segment encodes a protein with DNA cleavage activity where the protein has an amino acid sequence that has at least about 25% amino acid sequence identity with SEQ ID NO: 29. In a further embodiment, the protein is capable of cleaving, at a specific site, a single DNA strand in a duplex where the specific cleavage site is for example, CCA, CCG or CCT.

The isolated DNA segment may be further characterized as having a DNA sequence with at least about 40% DNA sequence identity with SEQ ID NO:28.

In another embodiment, the isolated DNA segment encodes a protein with DNA cleavage activity where the DNA segment has at least about 10 contiguous bases identical to sequences contained in SEQ ID NO:28. Preferably, a protein of this type is capable of cleaving, at a specific site, a single DNA strand in a duplex where the specific cleavage site is for example, CCA, CCG or CCT.

In another embodiment, the isolated DNA segment encodes a protein with DNA methylase activity, which shares at least about 47% amino acid sequence identity with SEQ ID NO:31. The sequence of the DNA segment shares at least about 53% DNA sequence identity with SEQ ID NO:30.

In additional embodiments, a recombinant nicking endonuclease is provided that has an amino acid sequence sharing at least about 25% sequence identity with SEQ ID NO: 29 and a recombinant DNA methylase, is provided which shares at least about 47% amino acid sequence identity with SEQ ID NO:31.

In an additional embodiment, a recombinant nicking endonuclease is provided wherein the endonuclease is a mutant having a deletion at the C-terminal end. For example, mutants with deletions of about 51 and 19 amino acid residues at the C-terminus end retain their specificity for CCA, CCG and CCT.

In an embodiment of the invention, a vector is provided that includes a segment of DNA, which has a sequence that has at least about 10 contiguous bases identical to sequences contained in SEQ ID NO:28. A host cell containing this vector is also provided.

In an additional embodiment of the invention, a method is provided for amplification of DNA, that includes the steps of incubating the DNA with a DNA polymerase capable of strand displacement and a recombinant nicking endonuclease (as described above) and obtaining amplified DNA. This amplification method can be performed isothermally. An additional amplification step may optionally be added to the method in which random primers and a strand displacement polymerase are added to the amplified DNA to enhance the yield of the amplification by another round of amplification. Examples of polymerases for use in the method include Bst polymerase, Thermomicrobium roseum pol I and E. coli DNA polymerase large (Klenow) fragment. An example of the recombinant nicking endonuclease is Nt.CviPII. The DNA may be obtained from a single bacterial colony.

In an embodiment of the invention, a method is provided for eliminating DNA from a sample of biological material, that includes (a) adding Nt.CviPII nicking endonuclease or mutant thereof to the sample of biological material; and (b) allowing the nicking endonuclease or mutant thereof to cleave the DNA so as to eliminate the DNA from the sample of biological material.

In an embodiment of the invention, a method for cloning a toxic nicking endonuclease is provided that depends on removing a C-terminal sequence from the DNA encoding the toxic nicking endonuclease; and cloning the truncated gene in a suitable host cell such as E. coli. This approach has worked effectively for toxic enzymes cloned from Chlorella viruses.

### BRIEF DESCRIPTION OF THE FIGURES

FIG. 1 A shows a cartoon of the CviPII nicking-modification system. This system contains 2332 nucleotides and two complete open reading frames (ORFs). The methyltransferase contains 8 motifs typically found in m5C DNA methyltransferases but lacks motif IX and X that are also typical of methyltransferases from organisms other than chlorella. The nicking endonuclease has two active site motifs that are characteristic of some restriction endonucleases.

FIG. 1 B shows a small amount of Nt.CviPII purified from E. coli analyzed by SDS-PAGE using 4-10% gel. Lane 1 is Nt.CviPII while lane 2 is a molecular weight marker.

FIG. 2A shows an alignment of the M.CviPII sequence (SEQ ID No I) with another chlorella methyltransferase (M.CviPI) sequence (SEQ ID. No. 2) and a bacterial methyltransferase (M.HhaI) sequence (SEQ. ID. No 3).

Motifs I through X of m5C methyltransferase are marked. Conserved residues in the motifs are indicated by dots. Sequences that are identical are shown in black boxes. Conserved but not identical residues are shown in grey boxes. Asterisks indicate catalytic residues and hashes indicate S-adenosyl-L-methionine binding residues.

FIG. 2B shows the amino acid sequences for Nt.CviPII (SEQ ID NO:4) and restriction endonuclease CviJI (SEQ ID NO:5). Sequences that are identical are shown in black boxes. Conserved but not identical residues are shown in grey boxes.

Two type II restriction endonuclease active site motifs (P-D/E-Xn-D/E/S-Z-K/E where Z=hydrophobic residue) are found in Nt.CviPII sequences namely (i) SerI26-AspI27-Xi₂-GluI39-IleI40-LysI41 and (ii) V189-Glu I90-X₂i-Glu21₁-Val212-Lys213. The latter motif can be partially aligned to the proposed active site of CviJI. Asterisks indicate conserved residues of the active site motif.

FIG. 3A shows the results of the experiment that probes the putative M-CviPII methylation site. The DNA is p\JC-cviPIIM expressing M-CviPII. The DNA was isolated and digested with MspI or ScrFI. The first cytosine at the Nt-CviPII cleavage site of CCD was shown to be methylated because the plasmid was cleaved by ScrFI but was resistant to MspI digestion.

FIG. 3B is a chromatogram showing target sequences for methylation by M-CviPII. PCR products derived from sodium bisulfide-treated p\JC-cviPIIM are compared to those from un-treated p\JC-cviPIIM. The change of cytosine to thymidine corresponds to unmodified cytosine, whereas the presence of cytosine in both sodium bisulfide-treated and un-treated DNA indicates C⁵-methylcytosine. Results for recognition sites CCT, CCG (SEQ ID NOS:8 and 9), CCA (SEQ ID NOS: 10 and 11), CCCG (SEQ ID NOS: 12 and 13) show that the first Cs are methylated in CCN triplets. The result for CCAA (SEQ ID. No. 14 and 15) show relaxed specificity towards the second C of CCAA. The modified nucleotides are indicated by down arrows.

FIG. 4A compares the results from IPTG-induced Nt.CviPII expression and un-induced cell extract under a Iac promoter on pUC19 DNA. Supernatant of the lysate of induced (upper panel) and un-induced culture (lower panel) from equivalent cultures were loaded on a SP FF column and eluted with linear gradient of 0.1-1 M NaCl. High DNA nicking activity was obtained with IPTG induction. N=relaxed circle, L=linear and S=supercoiled.

FIG. 4B shows that Nt.CviPII is surprisingly thermostable being active at least to temperatures of 60 C. 0.5 μg of pUC19 was incubated with 1 unit of Nt.CviPII for 1 hour at various temperatures (lanes 2-10). The reactions were stopped and analyzed by electrophoresis on 1.5% agarose gel. Lane 11 is linearized pUC19 DNA, lane 12 is supercoiled pUC19 DNA and lanes 1 and 13 are 100 base pair size markers.

FIG. 4C shows the results of Nt.CviPII-cleaved pUC19 and single stranded M13 phage DNA by electrophoresis on 6% poly-acrylamide gel with 7 M urea.

Lane 1: size marker.

Lane 2: pUC19 (double strand DNA) with 1 unit of Nt.CviPII.

Lane 3: M13 (single strand DNA) with 1 unit of Nt.CviPII.

Lane 4: M13 (single strand DNA) with 0.5 units of Nt.CviPII.

Lane 5: M13 (single strand DNA) with 0 units of Nt.CviPII.

Lane 6: LMW: low molecular weight. DNA size marker.

FIG. 5 shows that CCA and CCG are the preferred substrates (SEQ ID NOS: 18 and 19) for Nt-CviPII cleavage. Nt.CviPII does not cleave at CCC but instead cleaves at the overlapping CCA site (SEQ ID NO: 20). CCT shows low-level cleavage by Nt-CviPII (SEQ ID NO: 21).

The upper schematic shows the sequence that was nicked by Nt.CviPII and read as the reverse-complement by the sequencing primer. Therefore, TGG corresponds to CCA, CGG to CCG, GGG to CCC and AGG to CCT. Triplet sequences in boxes are CCN sites designed on the substrate DNA. Native CCA sequences of pUC19 are underlined. The arrowheads indicate the cleavage site. The arrows under the chromatographs and the bracketed “a” in the schematic indicate the adenine added by the template-independent activity of Taq DNA polymerase used in sequencing reactions at the cleavage site.

FIG. 6 shows a schematic outline of isothermal amplification using Nt.CviPII and Bst DNA polymerase I large fragment.

FIG. 7A shows that nicking-endonuclease mediated isothermal amplification of DNA prefers a DNA polymerase with strand displacement activity. Purified E. coli DNA was incubated with Nt.CviPII in combination with Bst DNA polymerase I large fragment (Bst), T. roseum (Tro), Vent DNA polymerase, T. aquaticus (Taq) or E. coli DNA polymerase I Klenow fragment (Klenow) in the presence of dNTPs. Only Bst polymerase and Tro polymerase amplified the DNA significantly. Klenow also amplified low levels of DNA. No detectable amplification was found using Vent or Taq or no polymerase.

FIG. 7B shows the results of isothermal amplification of purified DNA from T. thermophilus and λ phage. T. thermophilυs and λ DNAs were amplified with Nt.CviPII and Bst DNA polymerase I large fragment. The amplified products were analyzed by 1.5% agarose gel electrophoresis. Lanes 2 and 4 shows the results of amplification only in the presence of both enzymes. Lane 5 is a marker. Lanes 1 and 3 show results from an amplification reaction containing only Bst DNA polymerase I large fragment and no Nt.CviPII. No detectable DNA was amplified without Nt.CviPII.

FIG. 7C shows that on 10-20% polyacrylamide gel with 7 M urea, the amplified DNA from FIG. 7B are resolved into single-stranded DNA from <25 to over 500 nt.

FIG. 7D shows that DNA can be amplified from a single bacterial colony subjected to heat treatment to release the DNA. Only a fraction of DNA was amplified without heating the cells (lane 6).

Lane 1 is the heat-treated cells from a single colony incubated with Bst DNA polymerase I large fragment, CviPI and Nt.CviPII.

Lane 2 is the heat-treated cells from a single colony incubated with Bst DNA polymerase I large fragment, and Nt.CviPII.

Lane 3 is the heat-treated cells from a single colony incubated with Bst DNA polymerase I large fragment only.

Lane 4 is the heat-treated cells from a single colony incubated with Bst DNA polymerase I large fragment, MspI and Nt.CviPII.

Lane 5 is Bst DNA polymerase I large fragment, and Nt.CviPII with no DNA template.

Lane 6 is the non-heat treated cells from a single colony incubated with Bst DNA polymerase I large fragment, and Nt.CviPII.

FIG. 8 shows DNA nicking activity of two Nt.CviPII truncation mutants. Four-fold dilutions of NPN297 or NPN329 (1 unit, 0.25 units, 0.06 units and 0.02 units) were incubated with 1 μg of pUC19 DNA at 37° C. for 1 hour. The cleavage products were analyzed on a 1.5% agarose gel in TBE. Supercoiled (SC) and linearized (L) pUC19 DNA and a marker were included for comparison.

FIG. 9 shows the results of Nt.CviPII truncation mutant-mediated DNA amplification. NPN297 (0.25 units) was used in combination with Bst DNA polymerase I large fragment (2 units) in the presence of 0.2 mM dNTPs and designated amount of λ DNA. The reactions were carried out at 55° C. for 30 minutes.

FIG. 10 shows the efficient removal of genomic DNA by Nt.CviPII in reverse transcription reactions.

Lane M: 2-log DNA ladder (New England Biolabs, Inc., Ipswich, Mass.).

Lane 1: reverse transcription (RT) without M-MuLV reverse transcriptase;

Lanes 2 and 4: RT with M-MuLV reverse transcriptase;

Lane 3: RT without M-MuLV reverse transcriptase, but in the presence of 0.5 unit of Nt.CviPII;

Lane 5: RT without M-MuLV reverse transcriptase, but in the presence of 2 units of Nt.CviPII.

Figure H A shows the DNA sequence of the CviPINt. gene (SEQ ID NO: 28).

Figure H B shows the amino acid sequence of the CviPIINt. gene (SEQ ID NO: 29).

FIG. 12A shows the DNA sequence of the CviPIIM gene (SEQ ID NO:30).

FIG. 12B shows the amino acid sequence of the CviPIIM gene (SEQ ID NO: 31).

### DETAILED DESCRIPTION OF THE INVENTION

The cloning and expression of CviPII nicking-modification system, purification of Nt.CviPII, and its utility in generating DNA oligonucleotides for random DNA amplification are described here. A significant aspect of this work is overcoming a toxicity problem caused by the frequent DNA nicking activity of the enzyme so as to produce a sufficient amount of recombinant Nt.CviPII, for its use as a molecular biology reagent. Nt.CviPII is of interest for a number of reasons including its ability to recognize a three-base sequence (CCD; D=A, G or T) of double strand DNA at the 5′ end of the first C of the top strand. It is also a naturally occurring frequently cutting nicking endonuclease. These properties have been exploited in a number of ways including a method of primer independent isothermal DNA amplification.

The nicking endonuclease Nt.CviPII described here has been cloned from a chlorella virus referred to as NYs-I. The CviPII nicking and modification system was cloned and expressed in E. coli. Initially, the cviPIIM gene was cloned in E. coli by the methyltransferase selection method. The adjacent ORFs were sequenced directly from the viral DNA. A downstream ORF showed some amino acid sequence identity to a restriction endodnuclease CviJI (RĜCY) in a BlastP search of all known genes in GeneBank.

An ORF for Nt.CviPII was identified, amplified by PCR and transcribed and translated in an in vitro transcription and translation system. The cell free extract showed DNA nicking activity when it was compared to the native Nt.CviPII nicking endonuclease. However, it proved difficult to produce large amounts of protein for further characterization. Therefore, more efforts were made to express cviPIIM and cviPIINt genes in E. coli. E. coli expression host ER2683 was pre-modified by introduction of plasmid pUC-cv/PiTM, and the cviPIINt gene was cloned in the expression vector pR976, a low copy number plasmid with P_{tac} promoter. Extra codons were added to the 5′ end of cviPIINt gene to incorporate a tag of 6 histidine residues to facilitate purification of the protein.

To minimize the toxicity of the nicking endonuclease associated with its ability to nick DNA every 50-60 bp, the nicking endonuclease gene was positioned 18 nucleotides downstream of the ribosome-binding site to reduce the level of translation. Recombinant Nt.CviPII production was induced by the addition of IPTG to the culture media. Nt.CviPII was purified through affinity-tag purification and traditional chromatography such as metal-chelation chromatography, heparin FF and ion-exchange chromatographies. The purified Nt.CviPII revealed a nicking site preference of CCR>CCT (R=A or G) and was active through a wide temperature range with highest activity at a temperature range of 30-45° C. while retaining activity at 55° C. and 60° C.

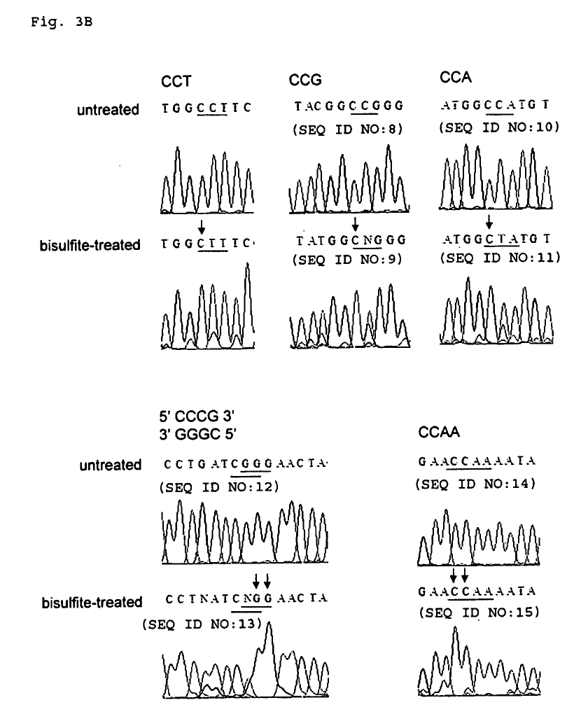

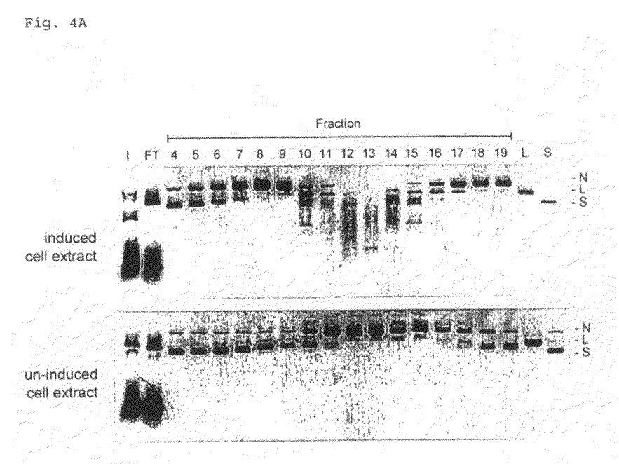

M.CviPII was shown to modify the first Cs in CCA, CCG, CCT and CCC triplet sequence (Example 1). It also modified the first two Cs in the sequence of CCAA (FIG. 3B).

To further increase the amount of Nt.CviPII obtained by recombinant technology, two C-terminal truncation mutants of Nt.CviPII fused to an intein and chitin-binding domain were generated. The combination of truncation and fusion decreased the toxicity of the nicking endonuclease to the host cells so that the fusion protein was over-expressed in E. coli strain ER2566 (New England Biolabs, Inc., Ipswich, Mass.). The fusion proteins were purified by chitin column chromatography and the fusion part was removed by self-cleavage activity of the intein induced by reducing agent. The cleaved Nt.CviPII truncation mutants were further purified by standard chromatographic steps. The truncation mutants of Nt.CviPII were found to possess the same sequence specificity but lower specific activity than the wild-type enzyme.

Uses of Nt.CviPII

(a) Isothermal Amplification

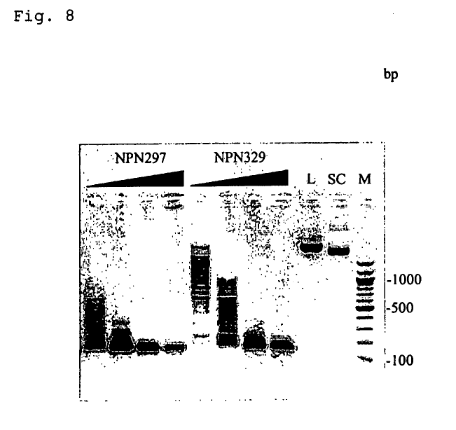

Due to the high frequency of Nt.CviPII cleavage sites and its partial duplex cleavage product, Nt.CviPII was used in conjunction with several DNA polymerases in isothermal random DNA amplification. An assay system was developed to determine conditions for isothermal amplification. This assay system is described in Example 3. Using this approach, it is possible to show amplification of a DNA using Nt.CviPII and DNA polymerases possessing strand-displacement activity. Moreover, FIG. 6 shows that DNA can be amplified from a single bacterial colony.

Nt.CviPII may also be used in prior art methods of isothermal amplification that utilize nicking endonucleases. These include: strand displacement amplification, exponential DNA amplification (EXPAR) or nick translation with DNA polymerases such as Klenow fragment, Bst DNA polymerase, Thermomicrobium roseum DNA pol I large fragment, or phi 29 DNA polymerase to replicate DNA at the nicked sites.

(b) Amplifying DNA from Single Colonies

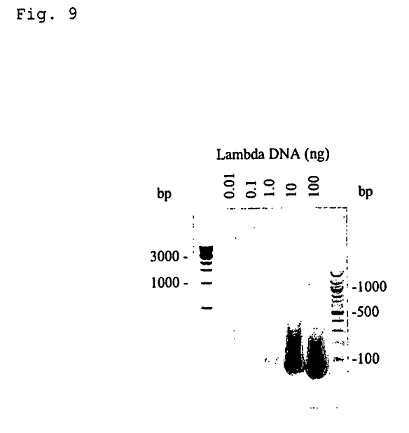

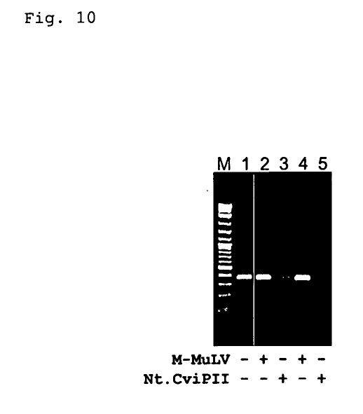

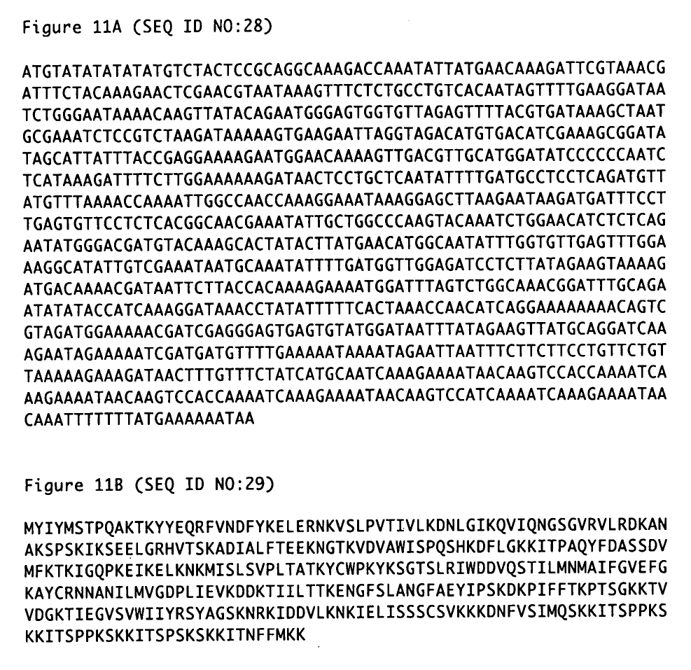

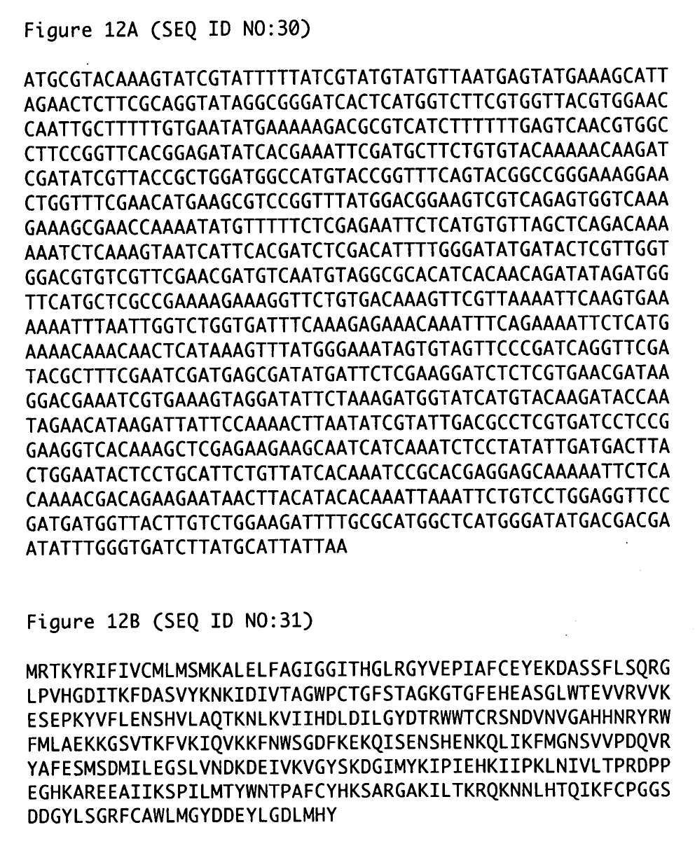

DNA can be amplified from a single E. coli colony using Nt.CviPII and a strand displacement DNA polymerase (FIG. 7D). The cells in the single colony have been broken by heat to release DNA but any other method in the art can be used where preferably no additional purification steps are required before performing amplification.

(c) Removing Genomic DNA from RNA or Protein Preparations

DNase I is the most commonly used enzyme in DNA contaminant removal from RNA samples. DNase I is a non-specific nicking endonuclease that works on single-stranded DNA, double-stranded DNA, and DNA-RNA hybrids. After DNAse I treatment, the enzyme must be removed from the RNA sample before other applications such as RT-PCR. However, DNase I is heat-resistant and therefore phenol extraction is required to remove DNase I completely (Aguila et al. BMC Molecular Biology 6:9 (2005)). In contrast, Nt.CviPII is a sequence-specific nicking endonuclease that recognizes double-stranded DNA only. Therefore, the DNA contaminant removal can be done by Nt.CviPII simultaneously with the reverse transcription reaction so that no extra purification steps are required. By choosing a different frequent nicking endonuclease, a different digestion pattern can also be achieved.

(d) Creation of Gaps for Assembling DNA Molecules and for Purifying DNA

The nicking endonucleases described herein may be used for creating single-stranded regions in duplex nucleic acids. Such single-stranded regions can take the form of gaps interior to the duplex, or terminal single-stranded regions. Single-stranded termini can be crafted to allow linkage of various elements via base-pairing with elements containing a complementary single-stranded region. This joining is useful, for example, in an ordered, oriented assembly of DNA modules to create cloning or expression vectors. This joining is also useful in attaching detection probes and purifying DNA molecules containing the single-stranded region. Gaps are useful in similar applications, including attaching detection or purification probes (U.S. Pat. No. 6,660,475 and U.S. Patent Publication No. 2003-0194736 AI herein incorporated by reference).

(e) Labeling DNA

The nicking endonucleases described herein can be used to label DNA. At first, nicks are introduced into DNA by Nt.CviPII. Then DNA polymerases with strand displacement activity can be used to replicate DNA. Radioactive dNTP, biotinylated dNTP, or dNTP with fluorophore modification can be added in the DNA extension reaction. The newly synthesized DNA should be labeled according to the dNTP used.

(f) Detecting Mutations

Similarly to restriction fragment polymorphism to detect genetic alterations, nicking fragment DNA polymorphism can be used to detect gene mutations if the point mutation takes place within the nicking site recognition sequence.

(g) Creating Relaxed Circles from Supercoiled DNA

The nicking endonucleases described herein can be used to prepare relaxed circular DNA under limited nicking conditions, e.g., using diluted Nt.CviPII. Supercoiled plasmid DNA is first nicked by Nt.CviPII provided that the plasmid contains at least one CCD site. The supercoiled DNA should be converted to nicked-open circular DNA, which can be gel-purified. The purified nicked DNA is treated with DNA ligase to generate relaxed circular DNA.

The references cited above and below as well as U.S. provisional application Ser. No. 60/620,939 are hereby incorporated by reference herein.

### EXAMPLE 1

### Cloning and Identification of cviPII M and cviPIINt

Chlorella virus NYs-I genomic DNA was digested partially with Sau3AI and ligated to a BamHI-digested and CIP-treated pUCAC (a derivative of pUC19 by inserting a PCR-amplified chloramphenicol resistant gene into AfIIII site of pUC19) and the ligated DNA was used to transform ER1992 competent cells to construct a Sau3AI genomic DNA library.

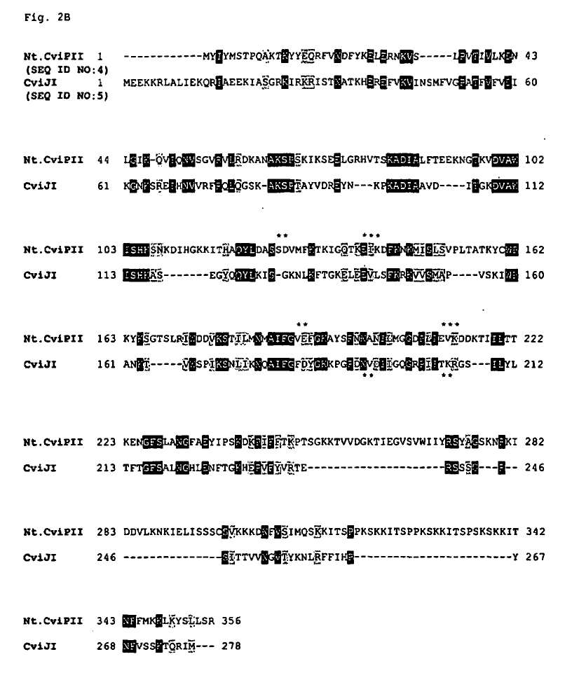

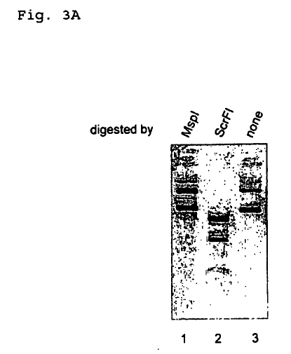

Approximately 10⁵ ampicillin resistant transformants were pooled and plasmid DNA was prepared. Clones that expressed M.CviPII methylase were selected by digesting pooled ampicillin and chloramphenicol resistant plasmids with MspI (cleaves CCGG and C^{m}CGG sequences but not ^{m}CCGG sequence). Eighteen plasmids from the Sau3AI genomic library were found to be partially resistant to MspI digestion. The inserts of six isolates were sequenced, which revealed an identical open reading frame (ORF, 1092 bp) that had 45.2% amino acid (aa) identity to the NYs-I encoded M.CviPI (recognition sequence GC) and 41% amino acid identity to chlorella virus IL-3A encoded M.CvUI (recognition sequence RGCY) (Xu et al. Nucl. Acids Res. 26: 3961-66 (1998); Shields et al. Virology 176: 16-24 (1990)) (FIG. 2A). The presence of most of the conserved motifs of m5C methylases in the primary structure of M.CviPII indicates that M.CviPII is a m5C methylase (Chan et al. Nucl. Acids Res. 32:6187-6. 199 (2004)).

The putative cviPIIM gene was amplified by PCR and ligated into pUC19 at the SphI and SaiI sites and transferred into E. coli ER2502. In vivo activity of M.CviPII was tested by challenging the plasmid isolated from ER2502 [p\JC-cviPIIM]. The plasmid was incubated with MspI (C^{A} CGG) or ScrFI (CĈNGG) at 37° C. for 1 hour in NEBuffer 2 (10 mM Tris-HCl, 50 mM NaCl, 10 mM MgCl2, 1 mM DTT, pH 7.9), New England Biolabs, Inc., Ipswich, Mass. The digested DNA was analyzed by agarose gel electrophoresis. The plasmid p\JC-cviPIIM was partially resistant to MspI but not to ScrFI (FIG. 3A). MspI cleaves C^{m}CGG, but not ^{m}CCGG, whereas ScrFI cleaves ⁿ¹CCNGG, but not C^{m}CNGG (http://rebase.neb.com/rebase). This indirect evidence (resistant to MspI digestion) indicated that M.CviPII modifies the first cytosine of the CCGG sequence, which is consistent with the result obtained from the native enzyme (Xia et al. Nucl. Acids Res. 16:9477-9487 (1988)).

To determine the site of modification, the plasmid pUC-cviPIIM used in methylase protection assay was treated with sodium bisulfide (EZ DNA Methylation Kit, Zymo Research Corporation, Orange, Calif.). The sodium bisulfide-treated DNA was purified by Qiaprep spin-columns (Qiagen, Valencia, Calif.) and used for PCR using primers that amplified the cviPIIM gene (MP-SphI-F and MP-SalI-F).

MP-SphI-F

(SEQ ID NO:7)

CGGTAAGCSATGCATGCGTACAAAGTATCGTATTTTTATCGTATGTATG
MP-SalI-R

(SEQ ID NO:22)

GAAGTCGACTTAATAATGCATAAGATCACCCAAATATTCGTCG

Untreated plasmid was also amplified by the same pair of primers as control. Methylated cytosines were protected from sodium bisulfide that converted un-modified cytosines to uracils, which are amplified as thymidines in PCR. Thus, by comparing with control PCR product, cytosine residues that became thymidines were un-modified while those that remained cytosines were methylated.

Sequencing of PCR-amplified DNA from the sodium bisulfide-treated pUC-cviPIIM indicated that most of the first but not the second cytosine of CCG, CCA and CCT are modified (FIG. 3B). Surprisingly, the first two cytosines in CCCG and CCAA were also modified (FIG. 3B, Table 2). In CCCG where CCC and CCG overlap, methylation of the first C in CCCG indicated that M.CviPII also methylates CCC site, a result of relaxed methyltransferase activity. Modification of the first and second Cs in CCAA suggested that M.CviPII modifies cytosine in relaxed recognition sequence under over-expressed condition (the cviPIIM gene was cloned in a high-copy-number plasmid and IPTG-induced).

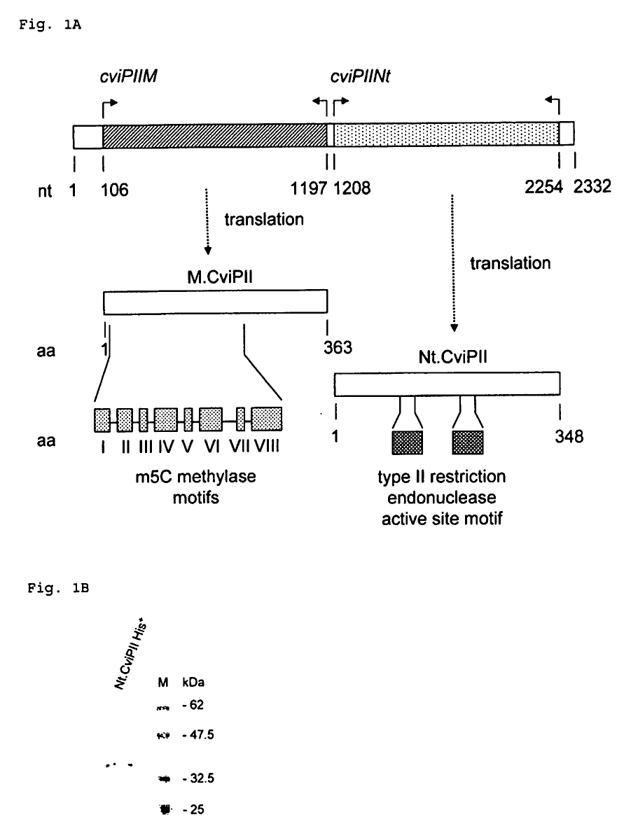

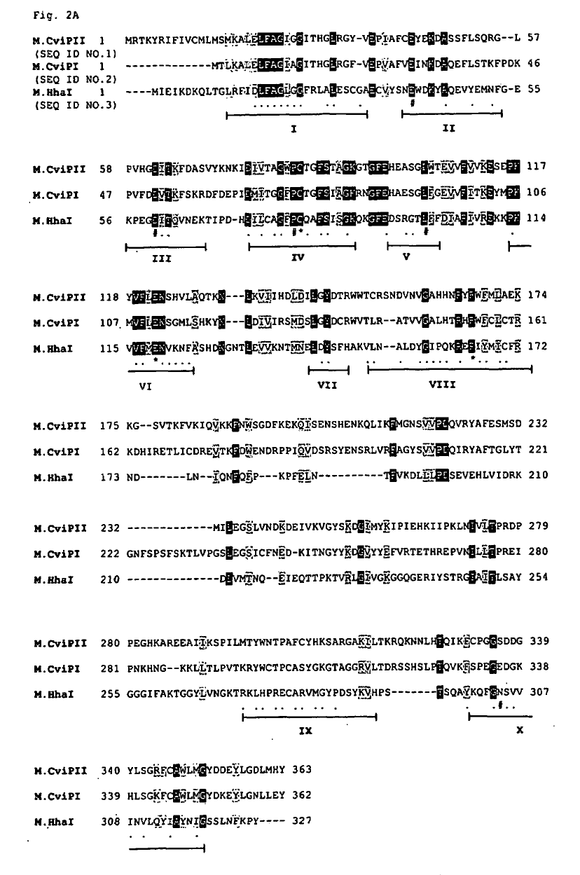

NYs-I DNA adjacent to cviPIIM gene was sequenced and an ORF of 349 codons was identified (in the same orientation) that began 12 nucleotides downstream of the cviPIIM stop codon (FIG. 1A). The protein expressed from the ORF had low but significant amino acid sequence identity to chlorella virus IL-3A encoded restriction endonuclease CviJI (RG/CY, with 18.3% identity (FIG. 2B)) which may reflect overlapping recognition sequence. The half site of CviJI (CY, Y═C or T) is similar to Nt.CviPII recognition sequence CCD. However, Nt.CviPII does not show significant similarity to other restriction endonucleases or nicking endonucleases with overlapping recognition sequence in GenBank. The pi value of Nt.CviPII was calculated to be 9.82 by ProtParam Tool of ExPASy Proteomics server of Swiss Institute of Bioinformatics.

### EXAMPLE 2

### Expression and Purification of Nt.CviPII

Due to the frequent Nt.CviPII nicking sites, difficulties in cloning the cviPHNt gene in E. coli were encountered. Initially, Nt.CviPII was expressed using in vitro transcription and translation system. A low level of nicking activity was detected in the lysate in comparison with the native Nt.CviPII. However, it was difficult to achieve a clear digestion pattern. To achieve sufficient enzyme for purification, the Nt.CviPII system was modified for expression in E. coli. The expression host ER2683 was pre-modified by expression of M.CviPII via introduction of pUC-cviPIIM. Additional measures were taken to construct a stable expression clone: (i) A low copy number plasmid pR976 with P_{tac} (pI5A replication origin) was used as the cloning vector for the cviPIINt gene; (ii) The cviPIINt gene was inserted 18 nucleotides downstream of the ribosome-binding site so as to reduce the expression level of the enzyme. The efforts to express M.CviPII in pACYC184 (pACYC-cwPJ/M gave marginal modification of host genomic DNA] and Nt.CviPII in pET21a vector failed to generate a stable expression clone.

The expression strain ER2683 [pUC-cviPIIM, pR976-cviPIINt] was successfully constructed. M.CviPII that was constitutively expressed under the control of lac promoter on pUCAC protected the host DNA from basal expression of the Nt.CviPII. The Nt.CviPII expression plasmid alone could not transform E. coli cells, indicating that the residual expression of Nt.CviPII in the absence of the cognate methyltransferase is lethal to the host. To determine the basal level of expression, induced or un-induced cultures of the expression strain ER2683 [pUC-cviPIIM, pR976-cviPIINt] were grown and partially purified by anion-exchange chromatography. The fractions were tested for DNA nicking activity.

A single colony of ER2683 [pUC-cw^{•PIZ} [symbol] ^{, pR}976-cviPIINt] was grown to mid-log phase in 2 liters of rich medium (10 g/l Tryptone, 5 g/l yeast extract, 5 g/l NaCl, pH adjusted to 7.2 with NaOH) containing Amp (0.1 mg/ml), kanamycin (Km, 0.05 mg/ml) and tetracycline (Tc, 0.01 mg/ml) at 30° C. at 280 rpm. One liter of culture was induced with 0.25 mM IPTG and the other was not. Both cultures were incubated at 16° C. for 18 hr and cells were harvested by centrifugation. The cell pellets (wet weight of 3.9 g for the induced culture and 5.0 g for the un-induced culture) were sonicated in 100 ml of 20 mM sodium phosphate, 0.1 M NaCl, pH 7.4. After centrifugation, the soluble fractions were loaded on a SP FF (25 ml bed volume, Amersham Biosciences, now GE Healthcare, Uppsala, Sweden) column and eluted with a linear gradient of 0.1-1 M NaCl. Two μl of the fractions were incubated with 0.5 mg of pUC19 at 37° C. for 1 hr. Reactions were stopped by adding 25 mM EDTA and analyzed by agarose gel electrophoresis. DNA nicking activity was distinguished from non-specific host nucleases. Non-specific nucleases produce a smear while Nt.CviPII produces a characteristic banding pattern of the digested DNA.

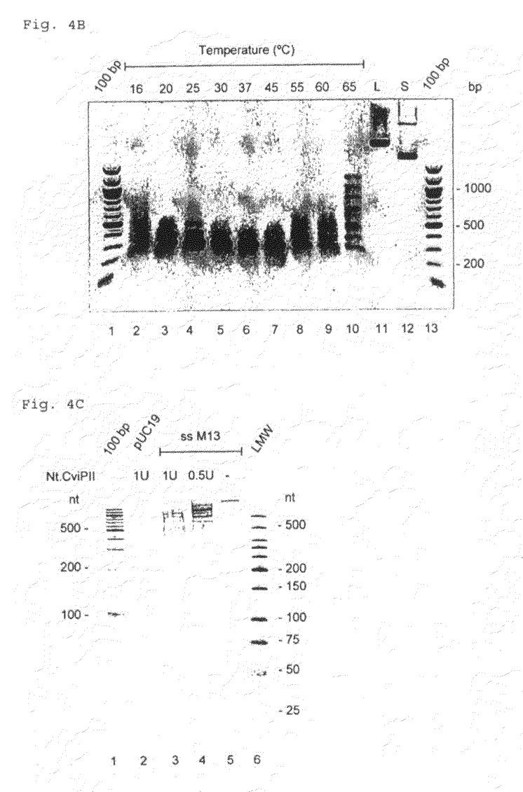

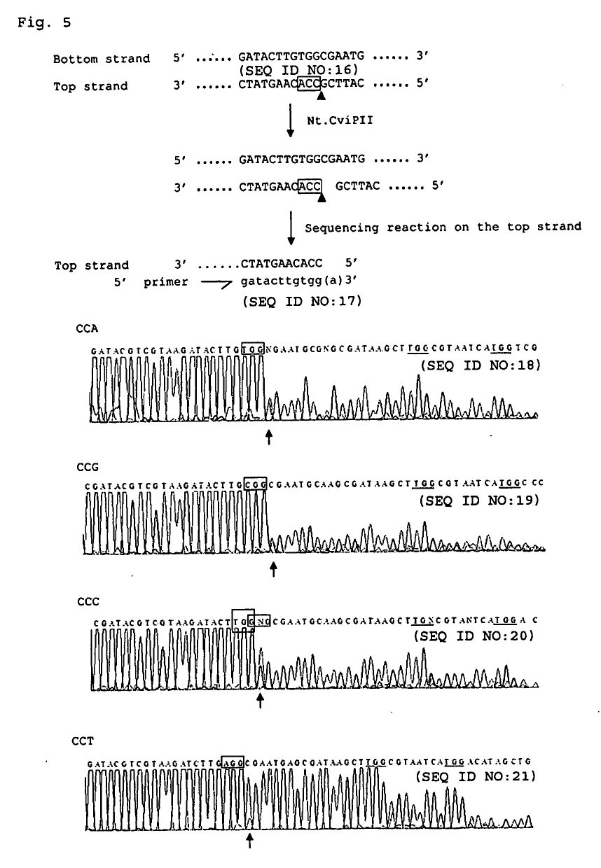

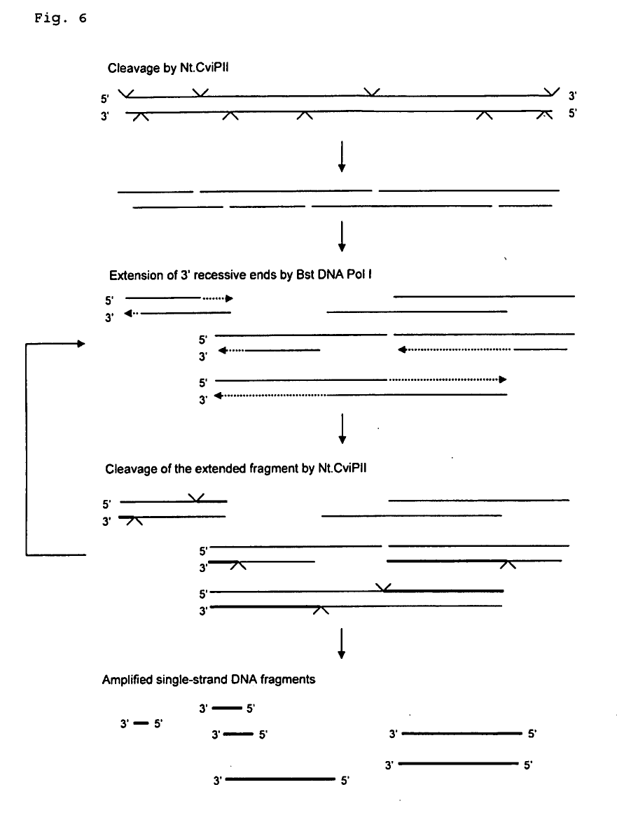

In the presence of IPTG, fractions eluting from a SP FF column exhibited significantly higher DNA nicking activity (FIG. 4A, top panel) than fractions from the un-induced culture (FIG. 4A, bottom panel). The un-induced culture, nevertheless, showed significant DNA nicking activity, indicating that there was basal expression of Nt.CviPII in the absence of IPTG. IPTG induction of cells grown at 16° C. produced more Nt.CviPII activity than cells grown at higher temperatures.

To purify the recombinant His-tagged Nt.CviPII, the supernatant obtained after sonication was loaded into a nickel-charged HisTrap column (HisTrap HP, 5 ml bed volume, Amersham Biosciences, now GE Healthcare, Uppsala, Sweden) and protein was eluted with a step gradient of 50, 100, 200, 300, 400 and 500 mM imidazole in 20 mM NaHPO4, 0.5 M NaCl, pH 7.4. DNA nicking activity was detected in fractions eluting in 50 mM and 300 mM imidazole. The 50 mM imidazole fraction was further purified on a heparin FF (25 ml bed volume, Amersham Biosciences, now GE Healthcare, Uppsala, Sweden) column and a SP FF (25 ml bed volume, Amersham Biosciences, now GE Healthcare, Uppsala, Sweden) column. The 300 mM imidazole fraction was concentrated and assayed. All purified protein preparations were concentrated by VIVASPIN 20 (10,000 MWCO, VIVASCIENCE) and stored in 10 mM NaHPO4, 250 mM NaCl, pH 7.0 with 50% glycerol at −20° C.

N-terminal sequencing of the purified protein eluted in 50 mM imidazole revealed a MSTPQAKTKYY sequence (SEQ ID NO:6), which corresponds to amino acids 5 to 15 in Nt.CviPII. Thus this fraction contains a protein initiated at the fifth codon of cviPIINt, which is an ATG. Mass spectrometry showed that the mass of this protein is 34, 110 Da, compared to the predicted value of 40,069 Da. This preparation was designated His⁻-Nt.CviPII.

Protein that eluted at 300 mM imidazole was concentrated and two bands of ˜34-36 kDa were observed by SDS-PAGE. N-terminal sequencing established that the upper band was Nt.CviPII with a 6-Histidine tag while the lower band was an E. coli 2-dehydro-3-deoxyphosphoheptonate aldolase contaminant. The two proteins were separated cleanly on a SP FF column, resulting in pure Nt.CviPII as judged by SDS-PAGE (FIG. 1B). This protein was designated HiS⁺-Nt. CviPII. Because this latter preparation was cleaner than His⁻-Nt.CviPII, it was used in all subsequent experiments.

Nt. CviPII Nicking Endonuclease Activity

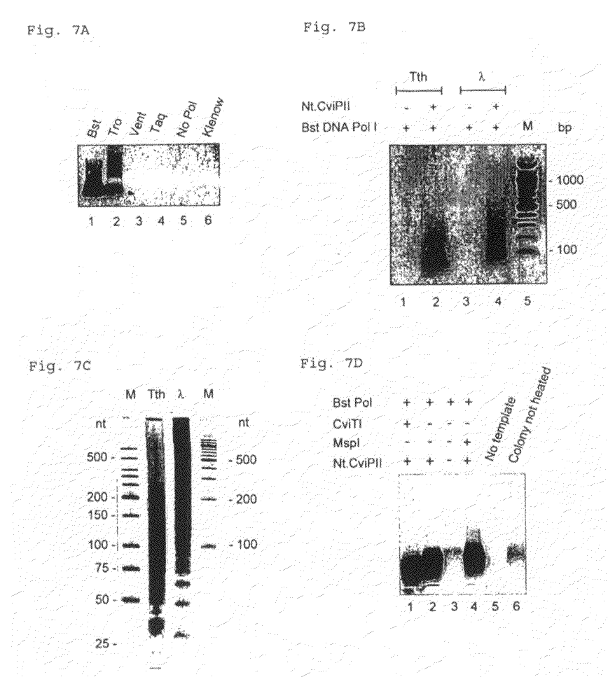

Nt.CviPII activity was measured at 16° C., 20° C., 25° C., 30° C., 37° C., 45° C., 55° C., 60° C. and 65° C. on 0.5 ug of pUC19 substrate in NEBuffer 4 (20 mM Tris-acetate, 10 mM magnesium acetate, 50 mM potassium acetate, 1 mM DTT, pH 7.9, New England Biolabs, Inc., Ipswich, Mass.) (FIG. 5A). DNA nicking activity was highest at 30-45° C., whereas the activity at 20-25° C. was higher than that at 55-60° C. Nt.CviPII showed lowest activity at 65° C., probably due to partial thermal denaturation. At 30-45° C., the cleavage product of pUC19 appears to be ˜200 bp in 1.5% agarose gel electrophoresis (FIG. 4B). A time course experiment at 37° C. established that a stable cleavage pattern was reached by 1 hr incubation. Longer incubation times or increasing the enzyme concentration did not alter the appearance of this pattern. Also, the pattern is not the result of Nt.CviPII inactivation at 37° C. because pre-incubation of Nt.CviPII at 37° C. for 90 min did not alter subsequent enzyme activity. Extended incubation of pUC19 with both batches of Nt.CviPII (HiS⁺ and His⁻) for 16 hours indicated that there is no significant contamination of non-specific nuclease. One unit of Nt.CviPII activity is defined as the amount of enzyme needed to cleave pUC19 into ˜200 bp products in 1 hr at 37° C., as judged by electrophoresis in 1.5% agarose gels. Protein concentrations were determined by the Bradford assay (Bio-Rad Protein Assay, Bio-Rad Laboratories, Hercules, Calif.) using BSA as a standard. The specific activity of HiS⁺-Nt. CviII is 9410 units/mg of protein.

Analyzing the pUC19 cleavage products by electrophoresis on poly-acrylamide gel containing 7 M urea indicated that the single-stranded DNA fragments were smaller than 150 nucleotides in size (FIG. 4C). There are 252 CCD sites on both strands of pUC19. With 2686 base pair and assuming that nicking sites are equally distributed in both strands, complete cleavage of pUC19 gives fragments of ˜20 nucleotides long. However, the end products of pUC19 are single-stranded DNA ranged from 25 to 150 nt as shown in FIG. 4B, suggesting that some of the sites are not cleaved. Therefore, some Nt.CviPII cleavage sites in pUC19 are apparently less susceptible to cleavage than others (see below).

The ability of Nt.CviPII to cleave single-stranded DNA was also tested. 250 ng of single-stranded M13 phage DNA was incubated with 0.5 or 1 unit of Nt.CviPII at 37° C. for 1 hour. Electrophoresis on poly-acrylamide gel containing 7 M urea showed that the single-stranded DNA was partially cleaved by Nt.CviPII. The low cleavage activity of Nt.CviPII on single-stranded DNA is likely due to the nicking activity on the transient duplex form of the phage DNA instead of the cleavage of single-stranded DNA per se.

### Nt.CviPII Cleavage Specificity

Double-stranded DNA substrates of 189 bp containing single CCA, CCT, CCC or CCG sites at nucleotides 160-162 (Table 1) were constructed by PCR. The substrates contain an internal XhoI site (ĈTCGAG) as a control to monitor cleavage. The substrate DNAs also contain EcoRI and HindIII sites at the 5′ and 3′ ends, respectively, for ligation into pUC19. The substrate DNA was ligated to pUC19 and the inserted DNA sequence was confirmed by sequencing. Two ug of pUC19-substrate was incubated with Nt.CviPII at 37° C. for 1 hour. Reactions were terminated by adding 25 mM EDTA and DNA samples were purified by QIAprep spin columns (Qiagen, Valencia, Calif.). One-eighth dilutions of the purified cleaved products were sequenced with custom primers that anneal at the 5′ end of the substrate DNA.

When a nick occurs in a double-stranded DNA, sequencing reaction on the nicked strand stops after the nicked base and the peaks in the sequencing chromatogram diminish sharply (Run-off sequencing). An extra adenine is added to the 3′ end of the newly synthesized single-stranded DNA due to the template-independent polymerase activity of Taq DNA polymerase used in the sequencing reaction. The adenine peak also helps identify the cleavage site. Consistent with a previous report (Xia et al. Nucl. Acids Res. 16:9477-87 (1988)), run-off sequencing showed that the DNA was cleaved 5′ of the first cytosine in CCA, CCG and CCT sequences, but not at the CCC site (FIG. 5). However, cleavage is less favorable at the CCT site than the CCA and CCG sites as only a small amount of the CCT substrate was cleaved (FIG. 5, bottom panel). A small peak of adenine following the AGG triplet indicates that some cleavage occurred at the CCT site. Cleavage occurred between the first and the second cytosine in the ĈCCA sequence. No cleavage took place before the first C in CCCA sequence. In all experiments, a minor peak corresponding to adenine signified the end of the template.

### EXAMPLE 3

### Truncation Mutants of Nt.CviPII: Cloning. Expression, and Purification of C-terminal Truncation Mutants of Nt.CviPII

DNA oligos were designed such that they served as primers for PCR amplification of two truncation mutants of Nt.CviPII. The primers also added NdeI and SapI sites at the 5′ and 3′ end of the amplified DNA, respectively, for cloning purposes: NPN297 (NP NdeI-F and NPN297-SapIR) and NPN829 (Np NdeI-F and NPN329-SAPIR).

NP-NdeI-F

(SEQ ID NO:23)

5′ ACCGTTGAGCAIAIGTATATATATATGTCTACTCCGCAGGCAAA
G 3′
NPN297-SapI-R

(SEQ ID NO: 24)

5′ GGTGGT TGCTCTTC CGCAACAGGAAGAAGAAATTAATTCTATTTT
ATTTTTCAAAACATCATCGAT3′
NPN329-SapI-R

(SEQ ID NO:25)

5′ GGTGGTTGCTCTTCCGCAGCATTTTGGTGGACTTGTTATTTTCTTTG
ATTTTGG 3′

Mutants NPN297 and NPN329 were generated such that they contains the first 297 aa (C-terminal deletion of 51 aa residues) and 329 aa (C-terminal deletion of 19 aa residues) of Nt.CviPII, respectively. The amplified DNA was ligated to pTXBI (New England Biolabs, Inc., Ipswich, Mass.) at NdeI and SapI sites. The mutant proteins were expressed as C-terminal fusion to intein Mxe GyrA followed by a chitin-binding domain. The ligated DNA was sequenced to confirm that there was no secondary mutation. The constructs pTXBI-NPN297 and pTXBI-NPN329 were transferred to E. coli strain ER2566 (New England Biolabs, Inc., Ipswich, Mass.) and grown in LB media or agar plates containing 100 μg/ml of Amp.

For purification of the truncated mutant nicking endonucleases, the following protocol was used for NPN297 and may also be used for NPN329. Single colony was inoculated to a starter culture of 100 ml of LB media containing 100 μg of Amp and grown at 37° C. with 250 rpm for 12-16 hours. Ten ml of the starter culture was inoculated to 1 liter of fresh LB media containing 100 μg of Amp for 6 liters of media. The culture was incubated at 37° C. with 250 rpm until OD600 reached 0.6-0.9. IPTG was added to the culture at a final concentration of 0.25 mM/L and incubation was continued for 3 hours at 37° C. with 250 rpm. The cultures were centrifuged at 3,550 g at 4° C. for 15 minutes. The cell pellets were stored at −20° C. The frozen cell pellets from each liter of culture were resuspended in 15 ml of 20 mM Tris-HCl, 0.5 M NaCl, 0.1% Triton X-100, 1 mM EDTA, pH 8.5 (chitin column buffer).

The resuspended cells were lysed by sonication on ice. The lysate was centrifuged at 26,700 g for 20 min at 4° C. The supernatant of the lysate was loaded to a chitin column (20 ml bed volume) at 4° C. After washing with 200 ml of chitin column buffer, the column was flushed with 40 ml of chitin column buffer with 40 mM of DTT within 10 minutes to induce cleavage of the intein. The column was incubated at 25° C. for 10-16 hours. The cleaved protein was collected by washing the column with 40 ml of chitin column buffer without DTT. The eluted protein solution was diluted one-fourth using 20 mM Tris-HCl, pH 7.7 such that the sodium chloride concentration decreased to ˜125 mM.

The diluted protein solution was loaded to a Heparin HyperD M column (BioSepra, Inc., Fremont, Calif.) bed volume=30 ml) at 4° C. The column was washed with 300 ml of a buffer containing 20 mM Tris-HCl, pH 7.7 and then with a linear gradient of 0-1 M NaCl in 20 mM Tris-HCl, pH 7.7. Fractions of 5 ml were collected and analyzed on SDS-PAGE. NPN297 was eluted in 0.6 M or higher concentration of NaCl. Fractions that contains the protein was pooled and dialyzed against 4 L of 10 mM potassium phosphate buffer, 50 mM NaCl, 1 mM EDTA, pH 7.0 (HTP column buffer) at 4° C. for 12-16 hours. The dialyzed protein solution was loaded to a hydroxyapatite column (Bio Rad Bio-gel HTP, bed volume=15 ml, Bio-rad Laboratories, Rockford, Ill.) at 2 ml/min. The column was washed with 150 ml of HTP column buffer and eluted by a linear gradient of 0-500 mM potassium phosphate buffer, 50 mM NaCl, 1 mM EDTA, pH 7.0. Fractions were collected for every 5 ml and analyzed using SDS-PAGE. NPN297 was found to elute at 0.25 M potassium phosphate. Fractions that contained NPN297 were pooled and dialyzed against 2 L of 40 mM Tris-HCl, 200 mM NaCl, pH 8.0 at 4° C. for 12-16 hours. The dialyzed protein solution was concentrated using VivaSpin concentrator (molecular weight cut-off=10 kDa). Equal volume of 100% glycerol was added to the concentrated protein solution. The protein preparation was stored at −20° C.

DNA Nicking Activity of the Truncation Mutants

DNA nicking activity and cleavage specificity of NPN297 and NPN329 were assayed essentially the same way as for the wild-type Nt.CviPII (Example 2). The truncation mutants were found to be active with the same cleavage specificity as the wild-type Nt.CviPII. The specific activity of NPN297 and NPN329 are estimated to be 2,600 units/mg and 1,400 units/mg, respectively, compared to 9,410 U/mg of the wild-type Nt.CviPIL Although the specific activity of the truncation mutants are lower than the wild-type Nt.CviPII, the yield of the truncation mutants are much higher such that the truncation mutant generates more units of activity from the same volume of culture than the wild type.

### EXAMPLE 4

### Isothermal Amplification of DNA Using Nt.CviPII and DNA Polymerases with Strand Displacement Activity

Because of the high frequency of cleavage sites and its single-stranded cleavage product, Nt.CviPII and NPN297 were used in conjunction with several DNA polymerases in isothermal random DNA amplification.

(a) Isothermal Amplification of Purified DNA

The following experiments were conducted with various purified DNA showing that the amplification method is generally applicable.

Two hundred ng of λ, E. coli or Thermus thermophilus genomic DNAs were incubated with 1 unit of Nt.CviPII and 16 units of Bst DNA polymerase large fragment, 8 units of Taq DNA polymerase or 4 units of Vent DNA polymerase in ThermoPol reaction buffer at 55° C. (20 mM Tris-acetate, 10 mM KCl, 10 mM (NhU)₂SO₄, 2 mM MgSO₄, 0.1% Triton X-100, pH 8.5), or 10 units of Klenow fragment of E. coli DNA polymerase I in EcoPol buffer (10 mM Tris-HCl, 5 mM MgCl₂, 7.5 mM DTT, pH 7.5) at 37° C. for 30 min supplemented with 0.1 mM dNTP. The amplified DNAs were analyzed by electrophoresis on either 1.5%-2% agarose gel or 6% polyacrylamide gel containing 7 M urea in I X TBE buffer.

E. coli DNA was incubated with Nt.CviPII and various DNA polymerases and dNTPs at various temperatures for 30 min. With the Nt.CviPII, Bst DNA polymerase I large fragment generated the highest yield of DNA at 55° C. The large fragment of DNA polymerase I from Thermomicrobium roseum (U.S. Pat. No. 5,962,296) also synthesized significant amounts of DNA while the addition of Taq DNA polymerases and Vent DNA polymerase did not result in any detectable DNA synthesis (FIG. 7A). Klenow fragment of E. coli DNA polymerase with Nt.CviPII generated a small amount of amplified DNA. Random DNA amplification can be achieved from T. thermophilus HB27 and λ DNA in the presence of Nt.CviPII/Bst DNA polymerase I large fragment but not with Bst DNA polymerase (PolI) large fragment alone (FIG. 7B). In FIG. 7C, the DNA having a PolI site in the range of <50 to 200 nucleotides efficiently amplified. The DNA mass was amplified approximately 50-fold with 10 ng input DNA generating ˜500 ng amplified small DNA fragments. The Nt.CviPII/Bst DNA pol amplified products can be further amplified by adding [N]₆, [N]₉, or [N]i₂ random primers together with Bst DNA pol large fragment to the amplification product by incubation at 50° C. for 60 min resulting in a second round of amplification which is expected to generate major DNA products in the range of 1 to 2 kb.

Although the amplification steps described above were done at 55° C., other temperatures can be used as long as denaturation of double-stranded DNA is favored. Nt.CviPII makes frequent cuts on the DNA and produces single-stranded products or partial duplex DNA with 5′ overhang (3′ recessed ends) (FIG. 6). Bst DNA polymerase I large fragment then fills in at the 3′ end until it reaches the end of the template. The extended DNA acted as a substrate for Nt.CviPII which, in turn, provided a new substrate for the polymerase, allowing linear amplification. The size of the amplified DNA can be increased or decreased by altering the amount (units) of Nt.CviPII in the amplification reactions.

From the collection of DNA polymerases tested, only Bst DNA polymerase I large fragment and Thermomicrobium roseum (Tro) DNA polymerase I large fragment produced significant amounts of amplified products. Klenow fragment also generated small amount of amplified DNA. These polymerases possess relatively high strand displacement activity among the polymerases tested. While not wishing to be limited by theory, strand displacement activity may be involved in removing the nicked fragment and revealing a recessive 3′ end for template-dependent amplification.

It was also demonstrated that incubation of random DNA oligonucleotides and fresh Bst DNA polymerase large fragment and dNTPs with the amplified product can result in DNA amplification. Alternatively, the amplified DNA can be purified and used as primers for direct amplification of genomic DNA through isothermal or thermocycling procedures.

Randomly amplified DNA has been used as a highly sensitive probe for arrays of DNA oligonucleotides carrying “signature sequences” of pathogenic biological agents such as E. coli 0157: H7 (Vora et al. Appl. Environ. Microbiol. 70: 3047-54 (2004)). The DNA amplification method presented here does not require synthesis of primers and can generate large quantities of single-stranded DNA from a single bacterial colony within a short time frame (e.g. 10 to 30 minutes).

Unlike rolling circle amplification that can be used to generate high coverage of the genome, this DNA amplification method may not necessarily cover the entire genome. The amplified DNA can be used as a probe to detect target DNA by Southern blotting.

The procedure can be adapted for environmental or clinical samples and labels such as biotin or fluorescein can be incorporated into the amplified product by using modified deoxy-nucleotides. Development of timely, sensitive and specific detection methods to identify important pathogens is of great importance in bio-defense and public health.

(b) Isothermal Amplification of DNA from a Single Bacterial Colony

Experiments were also performed to amplify DNA from a single E. coli colony using Nt.CviPII and Bst DNA polymerase I large fragment (FIG. 7D, lane 2). Single colonies of E. coli (˜0.2 mm in diameter) were suspended in 50 ml of water, heated at 94° C. for 8 min, centrifuged, and 20 ml of the supernatant was used for amplification. Heating the colony at 94° C. for 8 min was necessary to release the DNA for optimal amplification (FIG. 7D, lane 6). Including a degenerate 4-base cutting restriction endonuclease CviTI (RĜCY) in the amplification reaction generated slightly shorter amplified DNA fragments as judged by agarose gel electrophoresis (FIG. 7B, lane 1). However, including a 4-base cutting restriction endonuclease MspI (ĈCGG) made no difference in the size of the amplified product.

### EXAMPLE 5

### The use of frequent Nicking Endonucleases Such as Nt.CviPII and Nt.CviOXI to Eliminate Contaminant DNA from RNA Samples

Nt.CviPII recognizes ^{A}CCD (D=A, T, or G), which occurs at every ˜21 bp. Nt.CviQXI recognizes R̂AG (R=A or G), which occurs at every 32-64 bp. When used alone or together, the frequent nicking endonuclease(s) can degrade almost any larger DNA into very small pieces. An example of this application is to use frequent nicking endonucleases to remove genomic DNA contamination from RNA samples before reverse transcription and RT-PCR.

About 500 ng rat liver total RNA was mixed with 2 μl CIT₂₃VN (50 μM, New England Biolabs, Inc., Ipswich, Mass.) and 7 μl dH₂O. After denaturation at 70° C. for five minutes, it was left on ice. A 10 μl mix containing 100 mM Tris-HCl, pH 8.3, 150 mM KCl, 6 mM MgCl₂, 20 mM DTT, 8 units of M-MuLV (New England Biolabs, Inc., Ipswich, Mass.), 40 units of RNase inhibitors, 100 nmole dNTP, with or without 0.5 unit wild-type Nt.CviPII or 2 units of truncated Nt.CviPII was added. After one hour incubation at 42° C., 30 μl dH₂O was added to dilute the cDNA product into a 50 μl solution, from which 2 μl was used in 35-cycle PCR amplification using GAPDH-specific primers:

(SEQ ID NO: 26)

5′ TGCMTCCTGCACCACCAACT 3′ (forward primer)

(SEQ ID NO: 27)

5′ YGCCTGCTTCACCACCTT C 3′ (reverse primer)

Following RT PCR, eight μl was analysed on a 1% agarose gel (FIG. 10). In the presence of M-MuLV reverse transcriptase, the RT-PCR reaction gave a specific product (lanes 2 and 4). In the absence of M-MuLV reverse transcriptase, the RT-PCR reaction also gave a specific product due to the presence of contaminating genomic DNA in the RNA sample (lane 1). When 0.5 unit of Nt.CviPII was added to the RT reaction, a significant amount of genomic DNA contaminant was removed, leading to a much weaker band when M-MuLV reverse transcriptase was not added (lane 3). When two units of Nt.CviPII was added to the RT reaction, almost all genomic DNA contaminant was degraded, leading to a non-detectable signal when M-MuLV reverse transcriptase was left out (lane 5). The results indicated that Nt.CviPII can be included in the reverse transcription step to efficiently remove genomic DNA (template) contamination. (Similar results have been obtained using Nt.CviQXI.)

1. An isolated DNA segment encoding a protein with DNA cleavage activity, wherein the encoded protein has an amino acid sequence which has at least 25% amino acid sequence identity with SEQ ID NO:29.

2. An isolated DNA segment according to claim 1, having at least 40% DNA sequence identity with SEQ ID NO:28.

3. An isolated DNA segment according to claim 1, wherein the protein is capable of cleaving, at a specific site, a single DNA strand in a duplex.

4. An isolated DNA segment according to claim 3, wherein the specific cleavage site is selected from CCA, CCG and CCT.

5. An isolated DNA segment encoding a protein with DNA cleavage activity, the DNA segment having a sequence characterized by at least 10 contiguous bases identical to sequences contained in SEQ ID NO:28.

6. An isolated DNA segment according to claim 5, wherein the protein is capable of cleaving at a specific cleavage site on a single DNA strand in a duplex.

7. An isolated DNA segment according to claim 6, wherein the specific cleavage site is selected from CCA, CCG and CCT.

8. An isolated DNA segment encoding a protein with DNA methylase activity, wherein the protein has at least 47% sequence identity with SEQ ID NO:31.

9. An isolated DNA segment according to claim 8, wherein the DNA segment has a DNA sequence with at least 53% sequence identity with SEQ ID NO:30.

10. A recombinant nicking endonuclease, comprising an amino acid sequence with at least 25% sequence identity with SEQ ID NO:29.

11. A recombinant DNA methylase, comprising an amino acid sequence with at least 47% sequence identity with SEQ ID NO:31.

12. A recombinant nicking endonuclease according to claim 10, wherein the endonuclease is a mutant having a sequence truncation at the C-terminal end compared with a native nicking endonuclease.

13. A recombinant nicking endonuclease according to claim 12, wherein 51 amino acid residues at the C-terminus have been removed.

14. A recombinant nicking endonuclease according to claim 12, wherein the 19 amino acid residues at the C-terminus have been removed.

15. A recombinant nicking endonuclease according to claim 12, having a cleavage specificity selected from CCA, CCG and CCT.

16. A recombinant nicking endonuclease according to claim 12, having a substantially similar cleavage activity to the native endonuclease.

17. A vector comprising a segment of DNA, the DNA further comprising at least 10 contiguous bases identical to sequences contained in SEQ ID NO:28.

18. A host cell comprising the vector of claim 17.

19. A method for amplification of DNA, comprising:
(a) incubating the DNA with a DNA polymerase capable of strand displacement and a recombinant nicking endonuclease having at least 25% sequence identity with SEQ ID. No 29; and
(b) obtaining amplified DNA.

20. A method according to claim 19, wherein the nicking endonuclease is a mutant having a truncation at a C-terminal end compared with a corresponding native wild type nicking endonuclease.

21. A method according to claim 20, wherein 19 or 51 amino acid residues at the C-terminus have been removed.

22. A method according to claim 19, wherein the amplification is isothermal.

23. A method according to claim 19, further comprising: (c) subjecting the amplified DNA from (b) to an additional amplification step in the presence of random primers and a strand displacement polymerase to enhance the yield of the amplification.

24. A method according to claim 19, or 23, wherein the DNA polymerase is selected from Bst polymerase, Thermomicrobium roseum pol I and E. coli DNA polymerase large (Klenow) fragment and the nicking endonuclease is Nt.CviPII.

25. A method according to claim 19 or 23, wherein the recombinant nicking endonuclease is Nt.CviPII.

26. A method according to claim 19 or 23, wherein the DNA is obtained from a single bacterial colony, the DNA polymerase is selected from Bst polymerase, Thermomicrobium roseum pol I and E. coli DNA polymerase large (Klenow) fragment and the nicking endonuclease is Nt.CviPII.

27. A method for eliminating genomic DNA from a sample of biological material, comprising:
(a) adding Nt.CviPII nicking endonuclease or mutant thereof to the sample of biological material; and
(b) allowing the nicking endonuclease or mutant thereof to cleave the genomic DNA so as to eliminate the genomic DNA from the sample of biological material.

28. A method for cloning a toxic nicking endonuclease; comprising:
removing a C-terminal sequence from the DNA encoding the toxic nicking endonuclease; and cloning the truncated gene in a suitable host cell.

29. A method according to claim 27, wherein the host cell is E. coli.

30. A method according to claim 27, wherein the toxic nicking endonuclease is derived from a Chlorella virus.
